(19) United States
(12) Patent Application Publication
(10) Pub. No.: US 2010/0286038 A1
(43) Pub. Date: Nov. 11, 2010

(54) FORMULATION CONTAINING CYCLIN-DEPENDENT KINASE INHIBITING COMPOUND AND METHOD OF TREATING TUMORS USING THE SAME

(76) Inventors: Valentyn Antochshuk, Cranford, NJ (US); Anita Dabbara, Princeton Junction, NJ (US); Paul Kirschmeier, Basking Ridge, NJ (US); David A. Parry, Mountain View, CA (US); Mohammed Shameen, Nanuet, NJ (US); Siu-Long Yao, West Windsor, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/678,320

(22) PCT Filed: Sep. 16, 2008
(86) PCT No.: PCT/US08/10778

§ 371 Date: Jul. 19, 2010

Related U.S. Application Data
(60) Provisional application No. 60/994,785, filed on Sep. 21, 2007.
(60) Provisional application No. 60/999,469, filed on Oct. 17, 2007.
(60) Provisional application No. 61/053,784, filed on May 16, 2008.

(51) Int. Cl. A61K 38/18; A61K 31/519; A61P 35/02; A61P 35/00

(52) U.S. Cl. 514/7.6; 514/259.3

(57) ABSTRACT

This application discloses a novel formulation containing a 3-amino-4-substituted pyrazole derivative which has cyclin-dependent kinase inhibiting properties, and a method of treating tumors using the novel formulation.

This application claims the benefit of priority under 35 USC 119(e) of provisional patent applications U.S. Ser. No. 60/994,185 filed Sep. 17, 2007, U.S. Ser. No. 60/999,469 filed Oct. 17, 2007 and U.S. Ser. No. 61/053,784 filed May 16, 2008, the disclosures of which are hereby incorporated by reference in their entirety.

### FIELD OF THE INVENTION

This application discloses a novel formulation containing a 3-amino-4-substituted pyrazole derivative which has cyclin-dependent kinase inhibiting properties, and a method of treating tumors using the novel formulation.

### BACKGROUND OF THE INVENTION

Identification of any publication in this section or any section of this application is not an admission that such publication is prior art to the present invention.

The mammalian cell cycle is a non-redundant process that integrates extracellular signaling, DNA synthesis, and mitosis. Disregulation of cell cycle control is a hallmark of all human cancers and is frequently associated with aberrant activation/regulation of cyclin-dependent kinases, also termed herein for convenience, CDKs. The lack of cell cycle regulation in tumor cells is believed to increase the propensity of apoptosis in tumor cells compared to normal tissue. Accordingly, specific CDK inhibitors may preferentially kill tumor cells while inducing only reversible cell cycle arrests within normal proliferating compartments. Tumor cell apoptosis may be achievable without mutagenic effect to normal tissue. Therefore, identification of compounds inhibiting essential, rate-limiting activities of CDK2 and CDK1 presents an attractive therapeutic strategy for treatment and management of oncology indications.

Compounds having cyclin-dependent kinase inhibiting properties (CDK inhibitors) and which contain a 3-amino-4-substituted pyrazole derivative structure are described in U.S. Pat. No. 7,119,200 to Guzi et al. (herein, “the '200 patent” and herein incorporated by reference in its entirety), issued Oct. 10, 2006. Efficient syntheses of these CDK-inhibiting compounds, for example, the compounds of Formula I, are described in published U.S. application no. 2006/0281756 (the '756 publication, herein incorporated by reference in its entirety),

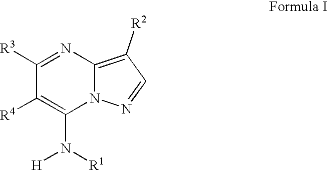

wherein, R¹, R², R³, and R⁴ are as defined in the '756 publication, which is incorporated herein in its entirety by reference.

The compounds of Formula I are useful in the therapy of proliferative diseases, for example, cancerous tumors.

As described in the '200 patent, which is incorporated by reference herein in its entirety, 2-(quinolin-5-yl)-4,5 disubstituted-azole derivatives of Formula I have been shown to have CDK inhibition activity with desirable IC₅₀ values using in vitro CDK2 inhibition tests and curve-fitting calculations. For example, the compound of Formula Ia, wherein, with reference to Formula I, R¹ is pyridin-3-yl-methylamine, R² is hydrogen, R³ is 1-methyl-piperazine, and R⁴ is hydrogen, has been shown to have a calculated IC₅₀ value of 5.6 micromolar for CDK receptor sites (see Table 87 in the '200 patent in Col. 706, Example 1).

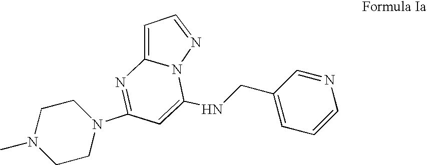

Other compounds having the structure of Formula I, for example, the compound of Formula II, wherein, with reference to Formula I, R¹ is 3-methyl-pyridine N-oxide, R² is ethyl, R³ is 2-piperidin-2-yl-ethanol, and R⁴ is hydrogen (see Example 1000 in the '200 patent beginning in Col. 667), also have useful CDK inhibition activity. The compound of Formula II is useful in inhibiting tumors, including, but not limited to, proliferation, angiogenesis and/or metastasis.

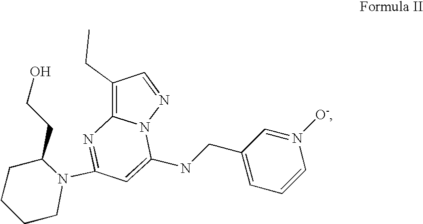

By way of example, the compound of Formula II may be useful in the treatment of a variety of cancers, including (but not limited to) the following: carcinoma, including that of the bladder, breast, for example breast cancer, colon, for example, colo-rectal cancer, kidney, bile duct, liver, lung, for example, small cell lung cancer and non small cell lung cancer, esophagus, gall bladder, pancreas, stomach, cervix, thyroid, prostate, ovarian cancer, vulvar cancer, peritoneal cancer, pseudomyxoma peritonei, and skin, for example, melanoma, and squamous cell carcinoma; hematopoietic tumors of lymphoid lineage, including leukemia, acute lymphocytic leukemia, acute lymphoblastic leukemia, chronic lymphocytic leukemia (CLL), B-cell lymphoma T-cell lymphoma, Hodgkins lymphoma, non-Hodgkins lymphoma [e.g., mantle cell lymphoma (MCL)], hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including, seminoma, teratocarcinoma, osteosarcoma, xeroderma pigmentosum, keratoacanthoma, thyroid follicular cancer and Kaposi's sarcoma.

### OBJECTIVES AND SUMMARY OF THE INVENTION

What is needed is a pharmaceutical formulation whereby compounds of Formula I having useful CDK inhibition activity can be administered to a patient in need of a therapeutic treatment in which administration of such compounds would be beneficial in the management or treatment of a disease state, for example, the management or treatment of tumors. These and other objectives and/or advantages are provided by the present invention.

One preferred aspect of the present invention is the provision of a concentrated pharmaceutical formulation containing the compound of Formula II,

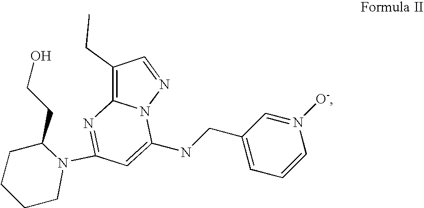

which can be adapted for administration to a mammal in need of treatment having a tumor responsive to the compound of Formula II, the formulation comprising the compound of Formula II, citric acid, sodium citrate and water at a pH of from about pH 2.5 to about pH 7.0. In one preferred embodiment, the buffered solution has a pH of from about 3.4 to about 5. In some embodiments it is preferred to use a mole ratio of the compound of Formula II: sodium citrate:citric acid of 1 mole of the compound of Formula II: 3.66 moles of sodium citrate:8.8 moles of citric acid. In some embodiments it is preferred for the formulation to comprise an aqueous solution containing on a wt./vol. basis: (a) 5 mg/ml of the compound of Formula II; (b) 21.4 mg/ml citric acid; and (c) 11.4 mg/ml sodium citrate dehydrate optionally admixed with hydrochloric acid and/or sodium hydroxide sufficient to adjust the pH of the concentrate to a pH of from about pH 2.5 to about pH 7.0, preferably the pH is adjusted to a pH of from about pH 3.0 to about pH 4.0, more preferably the pH is adjusted to about pH 3.5.

Another preferred aspect of the invention is an IV-infusible formulation comprising the compound of Formula II in a buffered solution having a pH of from about pH 2.5 to about pH 7.0, the formulation provides at least one PK behavior at a dosage indicated in accordance with those at shown in FIG. 2A and/or 2B and/or in Table V, when infused into a human at the indicated dosage levels. In one preferred embodiment, the buffered solution has a pH of from about 3.4 to about 5. Preferably, the formulation provides at least one of the AUC/dose curves illustrated in FIG. 2A and/or 3 when infused at the dosage level shown for each curve in a 2-hour infusion.

Another preferred aspect of the invention is an IV-infusible formulation comprising the compound of Formula II in a buffered solution having a pH of from about pH 2.5 to about pH 7.0, the formulation provides at least one PK behavior at a dosage indicated in accordance with those at shown in FIG. 2C and/or Table VI, when infused into a human at the indicated dosage levels. Preferably, the formulation provides at least one of the AUC/dose curves illustrated in FIG. 2C when infused at the dosage level shown for each curve in a 2-hour infusion. In one preferred embodiment, the buffered solution has a pH of from about 3.4 to about 5.

Another preferred aspect of the invention is the provision of a formulation comprising an aqueous organic acid, its conjugate salt, and the compound of Formula II which provides the PK profile shown in FIG. 2A, 2B, and/or 2C upon IV infusion into a human. Preferably the formula comprises a mole ratio of the compound of Formula II: organic acid:conjugate salt of: 1 mole of the compound:8.8 moles of organic acid:3.7 moles of conjugate salt. Preferably the formulation provides a PK for a two hour infusion of a dose comprising 1.85 mg/M² having a t_{1/2} of about 2.15 hours, a Cₘₐₓ of about 77 ng/ml, and/or an AUC of about 181 ng hr/mL. Preferably the formulation provides a PK for a two hour infusion of a dose comprising 3.7 mg/M² having a tᵢₙ of about 2.75 hours, a Cₘₐₓ of about 136 ng/ml, and/or an AUC of about 336 ng hr/mL. Preferably the formulation provides a PK for a two hour infusion of a dose comprising 7.4 mg/M² having a t_{1/2} of about 2.86 hours, a Cₘₐₓ of about 353 ng/ml, and/or an AUC of about 994 ng hr/mL. Preferably the formulation provides a PK for a two hour infusion of a dose comprising 14.8 mg/M² having a t_{1/2} of about 2.78 hours, a Cₘₐₓ of about 396 ng/ml, and/or an AUC of about 1053 ng hr/mL. Preferably the formulation provides a PK for a two hour infusion of a dose comprising 29.6 mg/M² having a t_{1/2} of about 2.79 hours, a Cₘₐₓ of about 1020 ng/ml, and/or an AUC of about 2531 ng hr/mL. Preferably the formulation provides a PK for a two hour infusion of a dose comprising 41.4 mg/M² having a t_{1/2} of about 2.8 hours, a Cₘₐₓ of about 1343 ng/ml, and/or an AUC of about 3041 ng hr/mL. More preferably the formulation provides a PK for a two hour infusion of a dose comprising 50 mg/M² having a t_{1/2} of about 2.9 hours, a Cₘₐₓ of about 1820 ng/ml, and/or an AUC of about 4050 ng hr/mL. Preferably the formulation provides a PK for a two hour infusion of a dose comprising 58 mg/M² having a t_{1/2} of about 3.2 hours, a Cₘₐₓ of about 1460 ng/ml, and/or an AUC of about 3290 ng hr/mL.

Table VIII is a cumulative representation of the data in Tables V, VI and VII with an expansion in patient numbers at certain doses as indicated in Table VIII. Another embodiment is the provision of a formulation comprising an aqueous organic acid, its conjugate salt, and the compound of Formula II which provides the PK profile shown in FIG. 2A, 2B, and/or 2C upon IV infusion into a human. Preferably the formula comprises a mole ratio of the compound of Formula II: organic acid:conjugate salt of: 1 mole of the compound: 8.8 moles of organic acid: 3.7 moles of conjugate salt. Preferably the formulation provides a PK for a two hour infusion of a dose comprising 1.85 mg/M² having a t_{1/2} of about 2.29 hours, a Cₘₐₓ of about 91 ng/ml, and/or an AUC of about 211 ng hr/mL. Preferably the formulation provides a PK for a two hour infusion of a dose comprising 3.7 mg/M² having a t_{1/2} of about 2.75 hours, a Cₘₐₓ of about 136 ng/ml, and/or an AUC of about 336 ng hr/mL. Preferably the formulation provides a PK for a two hour infusion of a dose comprising 7.4 mg/M² having a t_{1/2} of about 2.86 hours, a Cₘₐₓ of about 353 ng/ml, and/or an AUC of about 972 ng hr/mL. Preferably the formulation provides a PK for a two hour infusion of a dose comprising 14.8 mg/M² having a t_{1/2} of about 2.78 hours, a Cₘₐₓ of about 396 ng/ml, and/or an AUC of about 1050 ng hr/mL. Preferably the formulation provides a PK for a two hour infusion of a dose comprising 29.6 mg/M² having a t_{1/2} of about 2.98 hours, a Cₘₐₓ of about 890 ng/ml, and/or an AUC of about 2430 ng hr/mL. Preferably the formulation provides a PK for a two hour infusion of a dose comprising 41.4 mg/M² having a t_{1/2} of about 2.8 hours, a Cₘₐₓ of about 1300 ng/ml, and/or an AUC of about 2890 ng hr/mL. More preferably the formulation provides a PK for a two hour infusion of a dose comprising 50 mg/M² having a t_{1/2} of about 2.9 hours, a Cₘₐₓ of about 1820 ng/ml, and/or an AUC of about 4090 ng hr/mL. Preferably the formulation provides a PK for a two hour infusion of a dose comprising 58 mg/M² having a t_{1/2} of about 3.2 hours, a Cₘₐₓ of about 1460 ng/ml, and/or an AUC of about 3300 ng hr/mL.

In some embodiments it is preferred to prepare the concentrate by providing a solution containing an organic acid and its conjugate salt, dissolve the desired amount of the compound of Formula II therein, titrate the resulting solution with aqueous acid and aqueous base until the solution containing the compound of Formula II attains the desired pH, and optionally diluting the buffered solution with water or an aqueous solution, for example, a saline solution, to achieve the desired concentration of the compound of Formula II. In one preferred embodiment, the buffered solution has a pH of from about 3 to about 5. In some embodiments it is preferred to prepare the solution used to prepare the concentrate from an organic acid and conjugate salt selected from citric acid/sodium citrate and lactic acid/sodium lactate.

In one aspect the present invention comprises treating or managing a disease state by administering the concentrate, or a diluted form of the concentrate, to a mammal in need thereof, preferably by admixing the concentrate with the contents of a Normal Saline USP IV bag, preferably a standard 250 ml saline IV bag, to provide a medicament suitable for intraveneous infusion. In some embodiments it is preferred to admix a sufficient quantity of the concentrate to provide from about 0.92 micrograms of the compound of Formula II/ml of saline solution to about 372 micrograms of the compound of Formula II/ml of saline solution.

In some embodiments, an amount of the diluted concentrate is administered to a mammal in need of treatment equal to from about 0.33 mg/m² to about 58 mg/m² of the compound of Formula II, preferably from about 0.33 mg/m² to about 58 mg/m² of the compound of Formula II, more preferably from about 50 mg/m² to about 58 mg/m² of the compound of Formula II. In some embodiments it is preferred to administer about 50 mg/m² of the compound of Formula II. In some embodiments it is preferred to administer the diluted concentrate by IV infusion.

In some embodiments it is preferred to administer by IV infusion the amount of diluted concentrate necessary to provide the desired amount of the compound of Formula II over a period of from about 1 hour to about 24 hours. In some embodiments administering an IV infusion comprising a dose of from about 0.33 mg/m² of the compound of Formula II to about 58 mg/m² of the compound of Formula II it is preferred to administer the infusion over a period of about 2 hours. In some embodiments it is preferred to administer to a mammal in need of therapy an amount of diluted concentrate necessary to provide a dose of 14.8 mg/m² of the compound of Formula II at an infusion rate that yields a steady state plasma concentration of about 58 ng/ml, preferably at a rate that delivers the diluted concentrate over a period of 24 hours.

In certain preferred embodiments, compound of Formula II is administered in one or more cycles at the desired dose density. In one preferred embodiment, the compound of Formula II is administered in a 28-day cycle comprising once weekly administration of the compound of Formula II for three weeks with one week off. In another preferred embodiment, the compound of Formula II is administered in a 21-day cycle comprising administration at Day 1 followed by 20 days off.

### BRIEF DESCRIPTION OF THE FIGURES

FIG. 1 presents a graph estimating the concentration (ng/ml) versus time (hours) profile for a 24 hour infusion of a solution comprising 14.8 mg/m² of the compound of Formula II.

FIG. 2A presents a graph showing Cₘₐₓ and mean concentration/time profiles in volunteer human patients receiving one of 9 different 2-hour IV infusion-administered doses of the present formulation in an amount ranging from 0.33 mg/m² to 41.4.6 mg/m² of the compound of Formula II over a 12 hour time period.

FIG. 2B presents a graph showing mean concentration/time profiles in volunteer human patients receiving one of 9 different 2-hour IV infusion-administered doses of the present formulation ranging from 0.33 mg/m² to 41.4 mg/m² of the compound of Formula II over a 24 hour time period.

FIG. 2C presents a graph showing mean concentration/time profiles in volunteer human patients receiving one of 8 different 2-hour IV infusion-administered doses of the present formulation in an amount ranging from 1.85 mg/m² to 58 mg/m² of the compound of Formula II over a 12 hour time period.

FIG. 3 presents a graph showing exposure in healthy human volunteers for a series of doses of the present formulation containing an amount of the compound of Formula II ranging from 0.33 mg/m² to 41.4 mg/m² administered by 2 hour infusion IV.

FIG. 4 presents a graph showing the effects of various dosing regimes of the compound of Formula II on tumor volume suppression in A549 NSCLC mouse xenograft models.

FIG. 5 presents a graph showing the binding affinity of the compound of Formula II to Cyclin A/CDK2 complexes.

FIG. 6 presents a graph showing the inhibition effect of the compound of Formula II in A2780 mouse xenograft ovarian cancer model.

FIG. 7 presents micrographs of typical hair follicles harvested from nude mice exposed to dosages of the compound of formula II, demonstrating suppression of phospho-Rb within the proliferating epithelial cells of the basal epithelium and hair follicles.

FIG. 8 presents a graph showing concentration/time profiles for a patient administered by 2 hour IV infusion a dose of the compound of Formula II in the amount of 1.85 mg/m² as observed on days 1 and 15 of a 28 day dosing cycle.

FIGS. 9A and 9B present individual patient PK profiles over 24 hours after receiving IV infusion dosages of the compound of Formula II in the amounts of 29.8 mg/m² and 41.4 mg/m² respectively.

FIG. 10 presents data showing the effect of various dosage levels of the compound of Formula II on BrdU incorporation in ex vivo blood samples.

FIG. 11 presents a chart showing falling levels of biomarkers in a patient diagnosed with pseudomyxoma peritonei after receiving 6 cycles of IV infusion dosages of the compound of Formula II.

FIGS. 12A and 12B present data showing the effect of various dosage levels of the compound of Formula II on BrdU incorporation in ex vivo blood samples.

FIG. 13 presents data showing the percentage inhibition of BrdU incorporation at an 8 hour timepoint following exposure to various dosage levels of the compound of Formula II in ex vivo blood samples.

### DETAILED DESCRIPTION OF THE INVENTION

As mentioned above, compounds of Formula II (defined herein) have activity as useful pharmaceutical compounds having cyclin-dependent kinase inhibiting properties. In particular, the inventors have surprisingly found that the compound of Formula II, when administered as an IV formulation is provided in therapeutic levels. In vitro calculations indicate that the compound of Formula II inhibits the cyclin-dependent kinases CDK1 with a calculated drug affinity (IC₅₀) of 4 nM, CDK2 with calculated drug affinity (IC₅₀) of 1 nM, CDK5 with a calculated drug affinity (IC₅₀) of 1 nM, and CDK9 with a calculated drug affinity (IC₅₀) of 4 nM. Moreover, the inventors have surprisingly found inhibition of CDK2/CDK1 in tumor cells leading to cell cycle arrest and apoptosis in more than 100 tumor cell lines including the standard NCI panel. Additionally, the inventors have surprisingly found that exposure of xenograft models to plasma trough concentrations of less than 25 nM of the compound of Formula II for less than two hours results in tumor growth inhibition or regression. The inventors have surprisingly found that a formulation containing the compound of Formula II suitable for IV administration to humans to achieve beneficial plasma levels for the treatment or management of proliferative disorders can be prepared which formulation comprises a buffered aqueous solution of the compound of Formula II. The formulations comprising the compound of Formula II, methods of treating benign and proliferative disease states using the formulations of the invention containing the compound of Formula II, and examples are described next in greater detail.

### Preparation of an Iv Formulation Comprising the Compound of Formula II

One aspect of the present invention is a formulation comprising the compound of Formula II suitable for intravenous (IV) administration. The compound of Formula II is soluble in acidic media, but sparingly soluble in neutral and basic media. The inventors have surprisingly found that a suitable formulation can be prepared by dissolving the compound of Formula II in an acidic solution made by dissolving in water an organic acid suitable for incorporation into a buffered system, and then titrating the solution of the compound of Formula II and dissolved organic acid to a desired final pH using a suitable base and a suitable acid as necessary to adjust the pH.

Suitable acids are any organic that can provide an aqueous solution having a pH more acidic than about pH 7.0, preferably a pH at least as acidic as pH 3.5, and when titrated with a suitable base can provide a buffer solution having a pH in the range which includes from about pH 7 to about pH 2.5, and preferably can provide a buffer solution having a pH of about pH 3.5. Preferably, the organic acid selected is freely water soluble, for example, lactic or citric acid, more preferable, the organic acid chosen has more than one acidic proton, for example, citric acid.

The compound of Formula II, sparingly soluble in neutral water, is readily soluble in aqueous acid solutions. Accordingly, in some embodiments, in the preparation of the formulation of the invention it is preferred to initially prepare an aqueous acid solution, preferably an acid solution comprising at least about 0.111 M of the selected acid, dissolve therein a conjugate salt, for example, sodium citrate and sodium lactate, and then dissolve in the resulting buffer solution an aliquot of the compound of Formula II. After dissolution is complete the resulting solution is titrated to achieve the desired pH using an acid, for example hydrochloric acid, and base, for example, sodium hydroxide, in the amounts needed to bring the solution to the desired pH or within the desired pH range. Preferably the solution is more basic than about pH 2.5, more preferred is a solution having a pH of from about pH 3 to about pH 5. In one preferred embodiment, the solution has a pH from about pH 3.0 to about pH 4.2, more preferably from about pH 3.4 to about pH 3.6. Once the desired pH is attained, the volume of the buffered acid solution containing the API is adjusted with one or more aliquots of water to bring the solution to a volume that yields a solution providing about 5 mg/ml of the API. It will be appreciated that suitable solutions can be prepared under aseptic manufacturing conditions or sterilized after manufacturing.

It will be appreciated that the formulation of the invention may be prepared using other processes yielding a buffered acidic aqueous solution having the desired concentration of API and a pH of from about pH 2.5 to about pH 7.0, preferably from about pH 3 to about pH 5, more preferably from about pH 3.0 to about pH 4.2, without departing from the formulation of the invention.

### Method of Use

In one embodiment, the formulation of the invention is a concentrated formulation intended to be diluted as needed by addition of the concentrate into a standard IV bag containing 250 ml of 0.9% saline solution in an amount that provides the desired number of milligrams of API to effect treatment. It is within the scope of the invention to adjust the concentration of the formulation as necessary to provide a formulation sufficiently concentrated in API to accommodate in the dead space of a 250 mL saline bag an amount of the concentrate providing the desired number of mg of API. However, preferably the concentrate contains about 5 mg/ml and when needed in an amount in excess of that which can be accommodated within the dead space of a 250 ml saline bag, the amount of concentrate needed is divided into aliquots which are added to multiple saline bags for administration to the patient. As will be appreciated as greater or lesser amounts of the concentrated solution are introduced into the saline bag the pH will be altered. Preferably, the pH of the infusible solution is between pH 3.4 and pH 7, more preferably between about pH 3.4 and about pH 5. Slightly more acidic solutions may be obtained at high dilution levels of the concentrate, as will be appreciated, due to the lowered buffering ability of the concentrate with increasing dilution. It will be appreciated that any pH value compatible with the patient's physiology can be used as long as infusion time is adjusted to prevent or minimize damage to the vein in which the composition is infused.

In another embodiment, the formulation of the invention is an aliquot of a sufficient amount of the above-described concentrate diluted in an amount of a saline solution to provide an IV infusible solution containing a sufficient amount of the compound of Formula II to provide a therapeutic level of the compound of Formula II to a patient when infused into the patient.

Without wanting to be bound by theory, it is believed that typically up to about 58 mg/m² of the compound of Formula II is administered to a patient in need of therapy, although greater amounts and lesser amounts can be administered as desired. Preferably, about 50 mg/m² to about 58 mg/m² of the compound of Formula II is administered to a patient in need of therapy. In one preferred embodiment, about 50 mg/m² is administered to a patient in need of therapy.

The entire amount of concentrate diluted in saline solution is infused to a patient over a period of time, typically a period of from about 2 hours to about 24 hours. In some embodiments it is preferred to infuse the entire amount of the formulation to a patient over a 2 hour period. In other embodiments it is preferred to infuse the entire amount of the formulation to a patient over an 8 hour period. In yet other embodiments, to enable a longer steady-state plasma level of the API, it is preferred to infuse the entire amount of the formulation to a patient over a 24 hour period. In some embodiments it is preferred to administer a dosage density of about 50 mg/m²/3 weeks. In some embodiments it is preferred to administer this dosage density in a 28-day cycle, that is, as a 16.7 mg/m² dose once-a-week for 3 weeks followed by one week off. In some embodiments it is preferred to administer this dosage density in a 21-day cycle, that is, as a single 50 mg/m² dose every 3 weeks (i.e., administration at Day 1 followed by 20 days off). In other embodiments, it is preferred to administer a dosage density of about 58 mg/m²/3 weeks. In some embodiments, it is preferred to administer this dosage density in a 28-day cycle, that is, as a 19.3 mg/m² dose once-a-week for 3 weeks followed by one week off. In some embodiments, it is preferred to administer this dosage density in a 21-day cycle, that is, as a single 58 mg/m² dose every 3 weeks (i.e., administration at Day 1 followed by 20 days off). Greater or lesser amounts can be used. Administration cycles which provide regular doses, for example, a 28-day cycle comprising once weekly administration for three weeks with a week off, or a 21-day cycle comprising administration at Day 1 followed by 20 days off, can be employed to provide the desired dose density.

Due to the key role of CDKs in the regulation of cellular proliferation in general, the novel formulation(s) containing a CDK inhibitor of Formula II are believed to act as reversible cytostatic agents useful in the treatment of any disease process which features abnormal cellular proliferation, for example tumor growth, for example, in treating cancer, including solid tumors such as skin, for example, melanoma, breast, for example breast cancer, brain, colon and colorectal cancer, prostate cancer, for example, prostate cancer, gall bladder, thyroid, cervical carcinomas, testicular carcinomas, vulvar cancer, pseudomyxoma peritonei, peritoneal cancer, ovarian cancer, non small cell lung cancer (NSCLC), etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (mantle cell lymphoma), B-cell lymphoma, T-cell lymphoma, hairy cell lymphoma, Burkett's lymphoma, promyelocytic leukemia; Skin: malignant melanoma, basal cell carcinoma, squamous cell carcinoma, Karposi's sarcoma, moles dysplastic nevi, lipoma, angioma, dermatofibroma, keloids, psoriasis; Adrenal glands: neuroblastoma; and Other tumors for example: xeroderma pigmentosum, keratoacanthoma and thyroid follicular cancer.

In some embodiments a formulation of the invention comprising a CDK inhibitor of Formula II is used to treat non-small cell lung cancer (NSCLC), breast cancer, ovarian cancer, acute leukemias [e.g., acute myelogenous leukemia (AML) or acute lymphocytic leukemia (ALL)], chronic leukemias (e.g., CLL), non-Hodgkin's lymphoma (e.g., MCL), melanoma, and multiple myeloma.

In one embodiment, a formulation of the invention comprising a CDK inhibitor of Formula II is administered to a patient over an 8 hour period. By way of example, and not limitation, dosage ranges of a formulation of the invention comprising a CDK inhibitor of Formula II for an 8 hour infusion period may be from about 1.85 mg/m² to about 70 mg/m² and from about 15 mg/m² to about 30 mg/m². In some embodiments this dosage is administered as a 21 day cycle (administration on Day 1 followed by 20 days off).

In another embodiment, a formulation of the invention comprising a CDK inhibitor of Formula II is administered to a patient over a 24 hour period. By way of example, and not limitation, dosages ranges for a 24 hour period may be from about 1.85 mg/m² to about 100 mg/m², from about 50 mg/m² to about 80 mg/m² and from about 30 mg/m² to about 70.0 mg/m². In some embodiments this dosage is administered as a 21 day cycle (administration on Day 1 followed by 20 days off).

In another embodiment, solid tumors (e.g., breast, melanoma, ovarian) are treated by the methods described herein. By way of example, and not limitation, in this embodiment a dosage of a formulation comprising a CDK inhibitor of formula II from about 1.85 mg/m² to about 58 mg/m² (e.g., 20 mg/m², 30 mg/m², 40 mg/m², 50 mg/m²) is administered on a 21 day cycle (i.e., administration at Day 1 followed by 20 days off). The dosage may be infused over a 2 hour, 8 hour or 24 hour time period.

In an alternative embodiment, solid tumors (e.g., breast, melanoma, ovarian) are treated by the methods described herein in conjunction with growth factors (e.g. GCSF and GMCSF). By way of example, and not limitation, dosage ranges of a formulation of the invention comprising a CDK inhibitor of Formula II may be from about 0.33 mg/m² and from about 100 mg/m² in a 21 day schedule (administration on Day 1 followed by 20 days off). The dosage may be infused over a 2 hour, 8 hour or 24 hour time period.

In another embodiment, a dosage of a formulation of the invention comprising a CDK inhibitor of Formula II from about 1.85 mg/m² to about 100 mg/m² and from about 40 mg/m² to about 70 mg/m² is administered in a 21 day cycle (i.e., administration at Day 1 followed by 20 days off). The dosage may be infused over a 2 hour, 8 hour or 24 hour time period.

In yet another embodiment, a dosage of a formulation of the invention comprising a CDK inhibitor of Formula II from about 0.33 mg/m² to about 30 mg/m², from about 0.33 mg/m² to about 14 mg/m² or from about 7 mg/m² to about 12 mg/m² is administered in a 28 day cycle (i.e., dosage once a week for three weeks followed by one week off). The dosage may be infused over a 2 hour, 8 hour or 24 hour time period. In some embodiments hematologic cancers are treated. In other embodiments solid tumors are treated.

As used herein, treatment of cancer includes treatment of cancerous cells, including cells afflicted by any one of the above-identified conditions.

The formulations of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse. The formulations of the present invention may also be useful in inhibiting tumor angiogenesis and metastasis.

### EXAMPLES

Described next are various examples which illustrate the formulation and method of treatment of the invention. All animal experiments were carried out in accordance with good laboratory practices in accordance with the standards of the pharmaceutical industry. All reagents used in the preparation of formulations were USP or Food Grade.

### Example 1

### Preparation of a Formulation Containing the Compound of Formula II

Into a 500 L stainless steel vessel equipped with a stirring paddle was placed 400 kg water at ambient temperature (between 20 and 30° C.). Citric acid (10,682 g) was dissolved therein, with stirring. The vessel was stirred for 10 minutes maintaining ambient temperature and the addition equipment was rinsed with 0.2 kg additional water, which was added to the solution. When the citric acid dissolution was complete, stirring was continued and 5,710 g of sodium citrate were added to the solution with stirring until it had dissolved, maintaining ambient temperature. The addition equipment was rinsed with 0.2 kg additional water which was added to the citric acid/sodium citrate solution. The temperature of the solution was adjusted and maintained at a temperature between 20° C. and 30° C. Into the solution was added, with stirring, 2,585 g of the compound of Formula II (96.7% active, API, compound of Formula II prepared in accordance with the methods described in the above-referenced '756 publication). Stirring was continued until all of the added API was dissolved in the citric acid/sodium citrate solution. The addition equipment was rinsed into the solution with 1.2 kg of additional water. When dissolution was complete, the volume of the resulting solution was adjusted to 490 kg by addition of water, and the pH was adjusted by titration with 1 M sodium hydroxide solution, and 0.1 M HCl solution to yield a final pH of pH 3.5. The volume of the buffered solution was adjusted to 507 kg by adding water.

### Example 2

### Studies of Xenograft Cancer Model Tumors in Nude Mice

The efficacy of the formulation comprising the compound of Formula II prepared in Example 1 was examined in the A2780 xenograft ovarian cancer model. For all experimental models, maximum tolerated dose was defined as that dose, over the duration of the given schedule, which resulted in a 20% weight loss in subject mice.

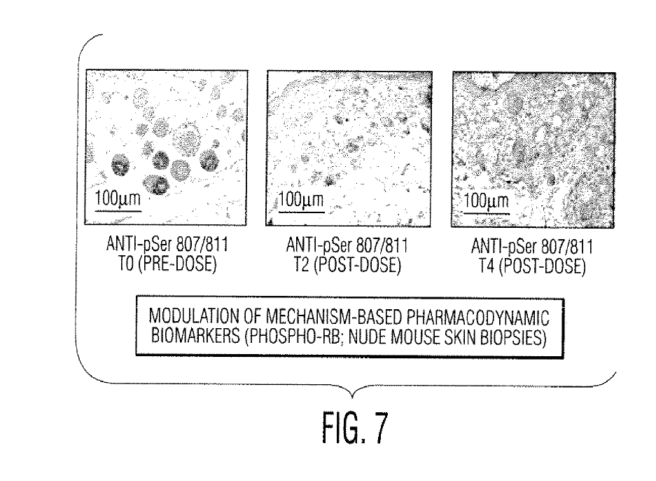

In carrying out this study, A2780 cells were inoculated into nude mice subcutaneously and tumors were permitted to grow until they had a volume of approximately 100 mm³ (approximately 7 days). Groups of ten of these test animals were injected IP with the formulation prepared in Example I at dosage levels of: 8 mg/kg (13% maximum tolerated dose (MTD)); 16 mg/kg (27% MTD); 32 mg/kg (53% MTD); and 48 mg/kg (80% MTD), daily for 10 days. A solution of paclitaxel was injected IP into a control group at a dosage level of 20 mg/kg twice weekly (50% MTD) along with control groups injected with formulation that did not include API. The results of inhibition of tumor growth are shown in FIG. 6. FIG. 6 shows that the control groups had tumor growth to a volume of about 800 mm³ over the ten day period, while animals that were dosed with paclitaxel had tumor growth inhibited by 63%. Groups of mice receiving formulation comprising the compound of Formula II exhibited tumor growth inhibition of 70% (both 8 mg/kg and 16 mg/kg dosing levels), 90% (32 mg/kg dosing level), and 96% (48 mg/kg dosing level). These data show that the 50% inhibition level (which in this model defines the minimum effective dose) is apparently less than 8 mg/kg. Moreover, it was found that animals exposed to the maximum dosage level of the compound of Formula II showed body-weight loss over the course of the experiment of about 5%, demonstrating that the formulation is well tolerated.

The formulation was administered to BALB/c mice at a dosage level of 40 mg/kg once per day for five days. Blood samples were obtained on days 1 and 7 following the last dose and analyzed on an Advia 120 hematology analyzer immediately after diluting 1:5 in PBS. A full differential blood count was performed, including red blood cell analysis (including reticulocyte, variant count, and hemoglobin analyses), white blood cell analysis (including differential lineage counts and peroxidase staining) and a thrombopoiesis analysis. No effect was observed on platelets or red blood cells.

In a related study, escalating doses of the formulation prepared in Example 1 and a formulation containing the compound of Formula III,

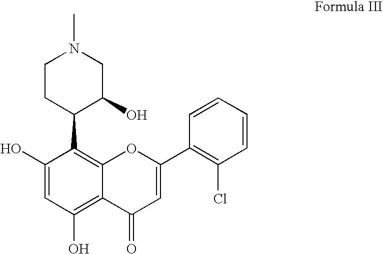

(Flavopiridol) were administered to nude mice bearing A2780 ovarian carcinoma xenografts on a once per day for 7 days dosing schedule. For this study, maximum tolerated dose was considered that dosage level inducing a 20% loss of body weight over the duration of the experiment and the minimum effective dose (MED) was defined as that dosage level inducing at least a 50% inhibition of tumor growth. The results are shown in Table I, which reports various therapeutic indices for each compound. The data in Table I indicate that the compound of Formula III was poorly tolerated yielding a nominal therapeutic index (MTD/MED) of less than 1, while the compound of Formula II was well tolerated and effective at a significantly lower dose than the MTD, the compound of Formula II yielding a nominal therapeutic index of greater than 10.

TABLE I

| | | In-Cell IC₅₀ | | | Therapeutic |
|---|---|---|---|---|---|
| | A/CDK2 | (Thymidine; | MTD* | MED* | index |
| Compound | IC₅₀ (μM) | μM) | (mg/kg) | (mg/kg) | (MTD/MED) |
| Formula III | 0.012 | 0.07 | <10 | 10 | <1 |
| Formula II | 0.001 | 0.004 | 60 | 5 | 12 |

*Dosage based on a dosing regime of once per day, intraperitoneal (IP).

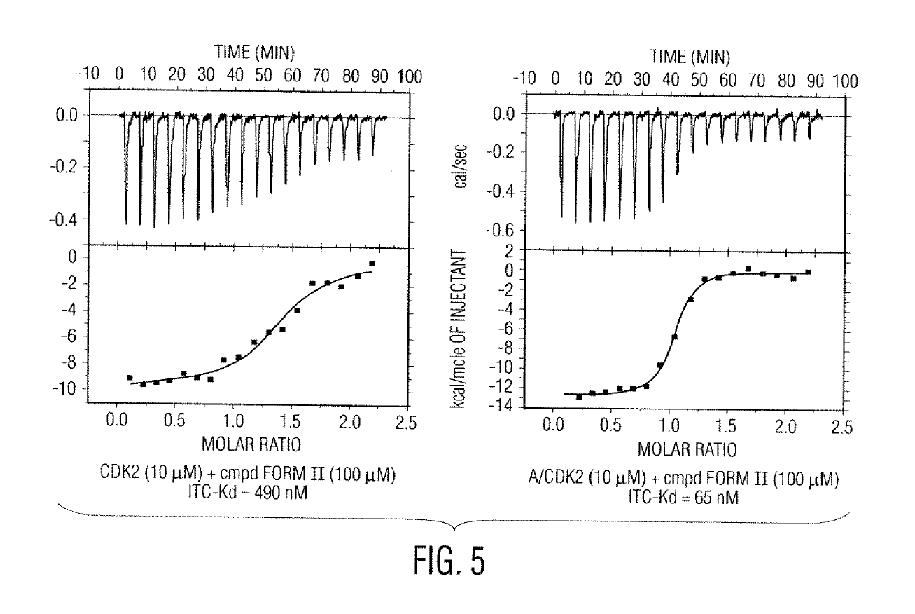

With reference to FIG. 4, nude mice bearing A549 NSCLC xenografts, established at a volume of greater than 100 mm³, were injected with a dosage equivalent to 260 mg/kg of the compound of Formula II using the formulation prepared in Example I over 5 different schedules shown: (a) 20 mg/kg once a day for 13 days; (b) 29 mg/kg for 3 days, followed by 2 days off for three cycles; (c) 52 mg/kg on days 1, 4, 7, 10, and 13; (d) 17 mg/kg thrice per day (equivalent to 52 mg/kg in 24 hours) on days 1, 4, 7, 10, and 13; and (e) 87 mg/kg on days 1, 7, and 13. As shown in FIG. 4, regression in tumor volume and mass was observed on all dosing schedules.

Similar studies were done using xenograft bearing mice for tumor type models of RPMI 8226, A549 NSCLC, NCI H460, PC3 (prostate), H82 (SCLC), SW527 (breast), and MDA MB231 (breast). The results, in comparison with the benchmark cytotoxic agents noted, are presented in Table II, below.

TABLE II

| | % inhibition | % inhibition | | |
|---|---|---|---|---|
| | observed | observed | % inhibition | |
| | at dosage of | at dosage of | observed | |
| | 5 mg/kg | 5 mg/kg | with benchmark | |
| Tumor Model | Formula II | Formula II | cytotoxic agent | |
| RPMI 8226 | 135 (regression) | 150 (regression) | 62 | (bortezomib) |
| A549 NSCLC | 73 | 160 (regression) | 0 | (paclitaxel) |
| NCI H460 | 45 | 90 | 5 | (paclitaxel) |
| PC3 (prostate) | 93 | 95 (regression) | 65 | (paclitaxel) |
| H82 (SCLC) | 56 | 65 | 40 | (carboplatin) |
| SW527 (breast) | 12 | 82 | 31 | (paclitaxel) |
| MDA MB231 | 52 | 70 | 68 | (paclitaxel) |
| (breast) | | | | |

Nude mice were given a single dose intraperitoneal of the formulation prepared in Example I equivalent to 40 mg/kg. Skin samples were harvested at various intervals following dosing and compared with biopsies harvested prior to dosing (T0). Biopsied samples were fixed overnight in 10% formalin. Samples were then placed in 70% alcohol for storage before being embedded in paraffin blocks, sectioned and stained with phospho-Rb Ser 807/811-specific antisera.

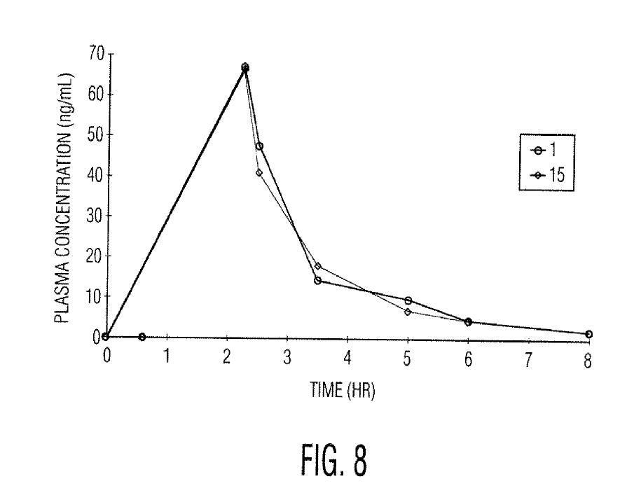

As shown in FIG. 7, which illustrates typical samples taken at intervals over several hours (T2 post dose) and (T4 post dose), the samples taken post-administration of the formulation indicate that the formulation induced rapid and sustained suppression of phospho-Rb within the proliferating epithelial cells of the basal epithelium and hair follicles.

With reference to FIG. 4, nude mice bearing xenografts were also dosed at the rate of 87 mg/kg once every six days, with the dosing schedule divided for single daily dose (20 mg/kg), single daily dose for 3 days followed by a 2 day off period (29 mg/kg single dose), single dose every three days (52 mg/kg, single dose), three 17 mg/kg doses in one day, repeated every three days, and one 87 mg/kg dose given every 6 days. Tumor regression was observed in animals treated with the compound of Formula II as compared with a control. The degree of tumor regression was similar for all dosing schedules examined.

### Example 3

### Inhibition of Thymidine Incorporation or Alamar Blue Viability in a Variety of Human Tumor Cell Lines and Confirmation of Cell Apoptosis Following Short Exposure to the Compound of Formula II

With reference to Table 4, various human tumor cell lines were exposed to increasing concentrations of the compound of Formula II for 24 hours and then assessed by inhibition of thymidine incorporation (adherent cells) or alamar blue viability assay (suspension cell lines). The level of activation of apoptosis was also assessed by determining the levels of activated caspase in each cell line. In each experiment, cells were exposed to increasing concentration of the compound of Formula II in cell medium for two hours by making multiple batches of the medium containing different amounts of the compound of Formula II and exposing different batches of cells to the medium containing the compound of Formula II at each particular concentration. The medium was removed and the cells were re-fed complete medium without the compound of Formula II present. The cells were evaluated fluorometrically for levels of activated caspase six hours post washout. Samples of each cell line were also subjected to SDS-PAGE and subsequently immunoblotted for p85 PARP caspase cleavage product. A positive apoptosis result was deemed if detectable activation of caspases and accumulation of p85 PARP was observed following a two hour exposure of a cell line to less than 0.1 micromolar concentrations of the compound of Formula II.

These data are presented in Table III. IC₅₀ values were calculated using the procedure described in the above-referenced '200 patent (Table 87 therein). The data in Table III indicate that all tumor types underwent cell cycle arrest in at least a portion of the different cell lines tested. Moreover, no tumor type tested exhibited particular sensitivity or resistance to the compound of Formula II. Additionally, these data show that caspase activation and apoptosis were detected in over 85% of the cell lines tested following a single exposure to the compound of Formula II. It should be mentioned that approximately 50% of the NCI-60 screening set are positive for MDR1 (multiple drug resistance Gene 1) gene expression.

TABLE III
Inhibition in a Variety of Cell Lines

| | | Caspase Activation |
|---|---|---|
| | | (number of sensitive |
| | Mean In-Cell IC₅₀ | cell lines out of |
| Tumor Type | (nanomolar) | total tested/single exposure) |
| Prostate | 12 | 4 of 5 |
| Breast | 8 | 6 of 7 |
| Colon | 17 | 5 of 9 |
| NSCLC | 14 | 8 of 9 |
| SCLC | 6 | 2 of 6 |
| Ovarian | 14 | 5 of 7 |
| Pancreatic | 15 | 11 of 15 |
| Melanoma | 9 | 9 of 9 |
| Leukemia | 6 | 5 of 6 |
| Bladder | 10 | 1 of 2 |
| Liver | 8 | 2 of 2 |
| Mantle Cell | 7 | 3 of 4 |
| lymphoma | | |
| Lymphoma | 7 | 8 of 8 |
| (NHL) | | |

Activation of apoptosis following short exposure times of A2780 cells to the compound of Formula II was confirmed by exposing A2780 ovarian cancer cell cultures to increasing concentrations of the compound of Formula II dissolved in growth medium for a period of two hours then removing the cells and re-feeding them in complete media without the compound of Formula II present for six hours (washout period). After the 6 hour washout period, cell extracts were prepared and evaluated by fluorometric assay for activated caspases (RFLU). In this study it was observed that caspase activity was reproducibly detected following 2 hours of exposure to the compound of Formula II present in as little as 0.05 micromolar concentration. When compared to cells treated with higher concentrations of the compound of Formula II (up to 5.0 micromolar) it was found that caspase activity did not increase. These data indicate that caspase activity is maximal at exposure levels of 0.05 micromolar.

To confirm that a two hour exposure profile was sufficient to induce durable effects on cell cycle distribution, asynchronously growing A2780 cells were exposed to concentrations of the compound of Formula II dissolved in growth media for two hours ranging from 0.001 micromolar to 30 micromolar in 16 batches, each approximately doubling the concentration of the compound of Formula II over the previous batch concentration. In each experiment, after exposure, the cells were washed out and re-fed growth media without the compound of Formula II present for 24 hours, then pulsed for 30 minutes with BrdU to establish the percentage of cells undergoing active DNA replication. Cells were then fixed, stained with an FITC-conjugated, anti-BrdU monoclonal antibody and propidium iodide. The cells thus prepared were analyzed using 2-color flow cytometry with doublet-discrimination. This study showed that at exposures of greater than 0.5 micromolar, the compound of Formula II completely suppressed DNA synthesis 24 hours after exposure. The samples exposed to concentrations of greater than 0.5 micromolar also showed accumulation of subG1 (associated with apoptotic cells). Escalation of exposure above 0.5 micromolar, up to concentrations of 30 micromolar did not augment the apoptotic phenotype.

Assessment of the mechanistic effects of short exposures of the compound of Formula II was carried out by exposing asynchronously growing A2780 cells to growth medium containing 0.1 mM of the compound of Formula II for two hours, washing out the cells and re-feeding the cells with medium that did not include the compound of Formula II. Following washout, lysates were prepared from the cells to assess the duration of the effects of exposure. Samples were separated on SDS-PAGE and immunoblotted with antisera specific for hypo-phosphorylated retinoblastoma protein and p85 PARP to assess inhibition of CDK's and activation of apoptosis. It was found that a 2 hours exposure of the cells to 0.1 mM of the compound of Formula II was sufficient to induce suppression of RB phosphorylation and caspase activation for a period exceeding 6 hours after the washout period.

Asynchronously growing A2780 cells were exposed to increasing concentrations of the compound of Formula II or the compound of Formula III for 16 hours. Cell lysates were separated on SDS-Page and immunoblotted with a rabbit polyclonal antisera specific for a CDK phosphorylation site (Ser 07/811) on the retinoblastoma tumor-suppressor protein (RB, a known CDK substrate). Accumulation of the p85 PARP product was also monitored to assess activation of caspases and correlate inhibition of CDKs with the onset of apoptosis. Consistent with the study described above in cell data assessing inhibition of thymidine incorporation (IC₅₀ of 4 nM), the formulation comprising the compound of Formula II suppressed phospho-Ser 807/811 following exposure. Phospho-Ser 807/811 was undetectable in lysates exposed to concentrations of greater than 6.25 nM in the compound of Formula II, and it was observed that complete suppression of detectable RB phosphorylation was required for the onset of apoptosis, indicated by the detectable p85 PARP. In comparison, exposure to the compound of Formula III (flavopiridol) did not induce significant suppression of RB phosphorylation, requiring concentrations of about 1 micromolar to induce detectable effects on phospho-Ser 807/811 levels. At concentration levels below those necessary to observe inhibition of RB phosphorylation, activation of apoptosis was observed. It was also observed that there was poor correlation between IC₅₀/EC₅₀ for the compound of Formula III in various in-cell assays of thymidine incorporation and mechanism based biomarkers, for example, phospho-RB.

Table IV indicates that the compound of Formula II is selective for the CDK family of Ser/Thr kinases, as determined by Upstate kinase Profiler counter-screening, whereas the compound of Formula III has more general activity, including the inhibition of c-Src tyrosine kinase.

TABLE IV

| | Cmpd. Form. II | Cmpd. Form. II | Cmpd. Form. III | Cmpd. Form. III |
|---|---|---|---|---|
| Kinase | at 1 μM* | at 10 μM* | at 1 μM* | at 10 μM* |
| AMPK | 94 | 82 | 67 | 20 |
| Blk | 69 | 100 | 73 | 26 |
| CaMKII | 102 | 91 | 44 | 30 |
| CDK6/cyclinD3 | 12 | 3 | 25 | 6 |
| CDK7/cyclinH/MAT1 | 7 | 4 | 18 | 6 |
| cSRC | 117 | 111 | 3 | 4 |
| Fes | 87 | 91 | 44 | 13 |
| Lyn | 108 | 110 | 73 | 18 |
| MSK1 | 94 | 97 | 74 | 37 |
| PKCα | 96 | 94 | 66 | 20 |
| PKCβII | 110 | 113 | 75 | 26 |
| PKCε | 93 | 88 | 45 | 9 |
| PKCθ | 95 | 95 | 23 | 5 |
| Rsk2 | 104 | 76 | 65 | 18 |
| YES | 92 | 98 | 68 | 40 |

*% remaining activity

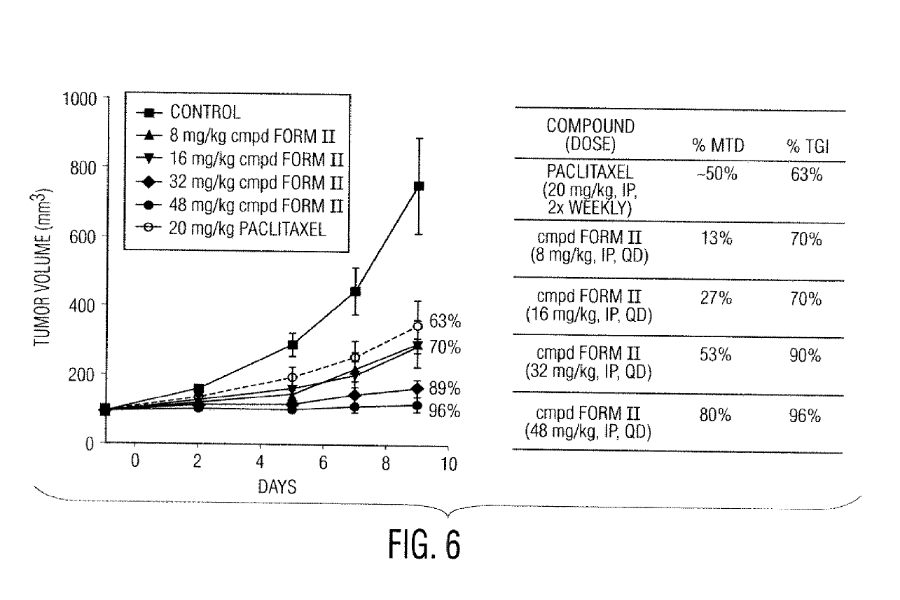

FIG. 5 also shows that the compound of Formula II binds with high affinity to cyclin A/CDK2 complexes.

### Example 4

### Studies in Human Volunteers

The formulation prepared in Example 1 was infused into volunteer human cancer patients after diluting it in a 250 ml standard 0.9% saline IV bag. The contents of the IV bag were infused over a two hour period. Dosages were prepared using an amount of the formulation providing doses of 0.33 mg/m², 0.66 mg/m², 1.32 mg/m², 1.85 mg/m², 2.59 mg/m², 3.63 mg/m², 5.08 mg/m², 7.11 mg/m², 10.00 mg/m², 12.00 mg/m² and 14.00 mg/m² of the compound of Formula II for the patient infused. Each patient was dosed on a 28 day cycle, comprising an infusion once a week for the first three weeks of the cycle on days 1, 8, and 15 of each cycle, with no dose administered in the week following the third dose. Plasma samples were collected prior to the start of an infusion, and at hours 1, 2 (completion of infusion), 2.25, 2.5, 3.0, 3.5, 4.0, 5.4, 6.0, 8.0, and 24 after the beginning of the infusion. Volunteer human cancer patients were also dosed once every three weeks (i.e., on a 21-day cycle) via a 2 hour IV infusion with 1.85 mg/m², 3.7 mg/m², 7.4 mg/m², 14.8 mg/m², 29.6 mg/m², 41.4 mg/m², 50 mg/m², and 58 mg/m². PK and AUC results obtained were similar between the 21-day dosing schedule and the comparable 28-day dosing schedule described above.

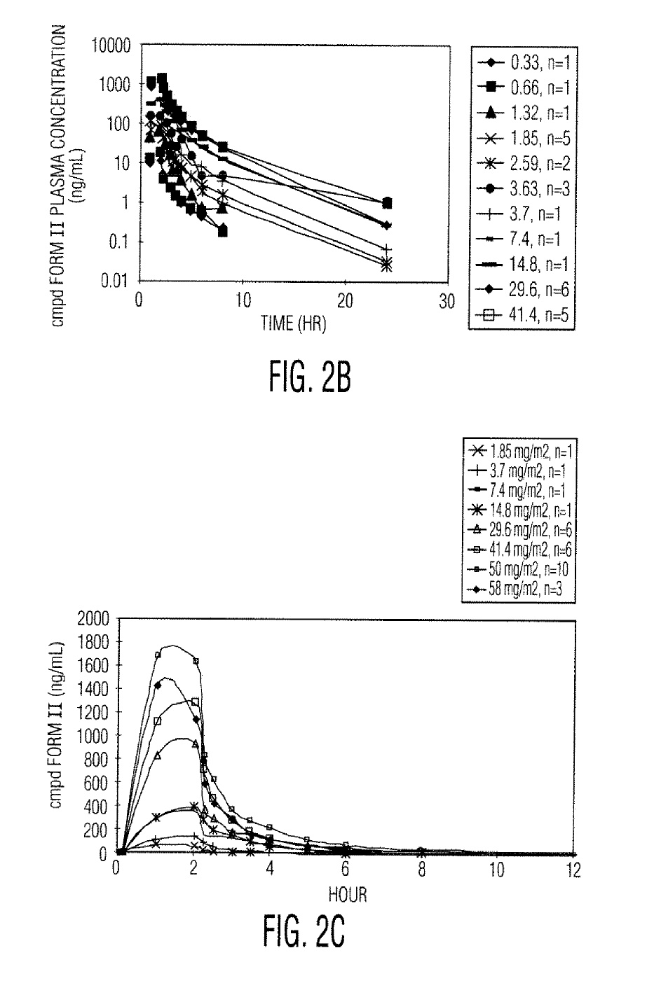

With reference to FIGS. 2A, 2B, and 2C, which show the plasma concentration in ng/ml of the compound of Formula II following commencement of a two hour infusion for the various dosage levels indicated, for each infusion, Cₘₐₓ is achieved between 1 and 2 hours after beginning the infusion. When the infusion is complete it can be seen that in each case the compound of Formula II is rapidly eliminated from plasma, with T_{1/2} ranging from about 1.4 to about 3.3 hrs depending on dosage.

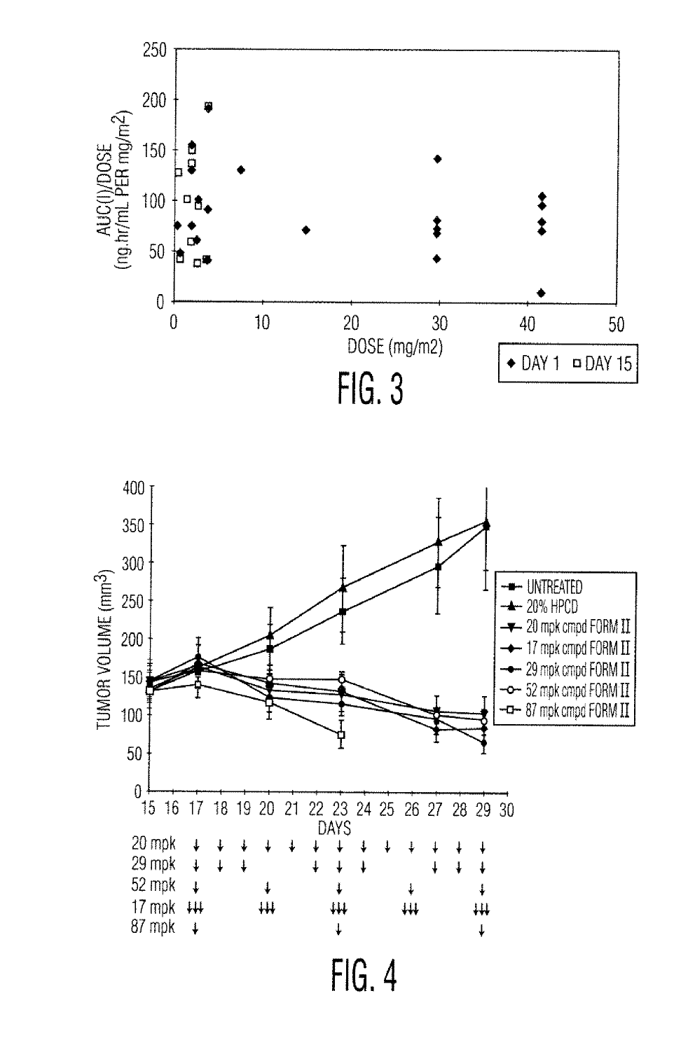

With reference to FIG. 3, which presents AUC (ng hr/mL)/dose (mg/m²) for the various doses administered on day 1 (diamonds) and day 15 (squares) of a 28 day dosing cycle, it appears that dose-proportional increases in exposure are observed for dosages of from 0.33 mg/m² to 41.4 mg/m², and that clearance is dosage independent. FIG. 2B also shows that plasma levels of 25 nM (attained at plasma concentrations of 10 ng/mL) are available from 2-hour infusions at dosages of greater than 1.32 mg/m².

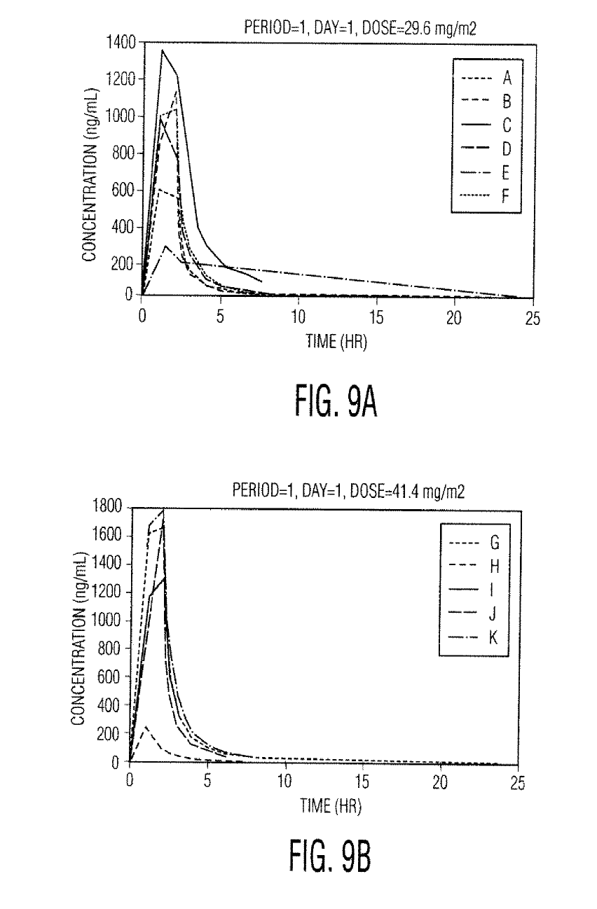

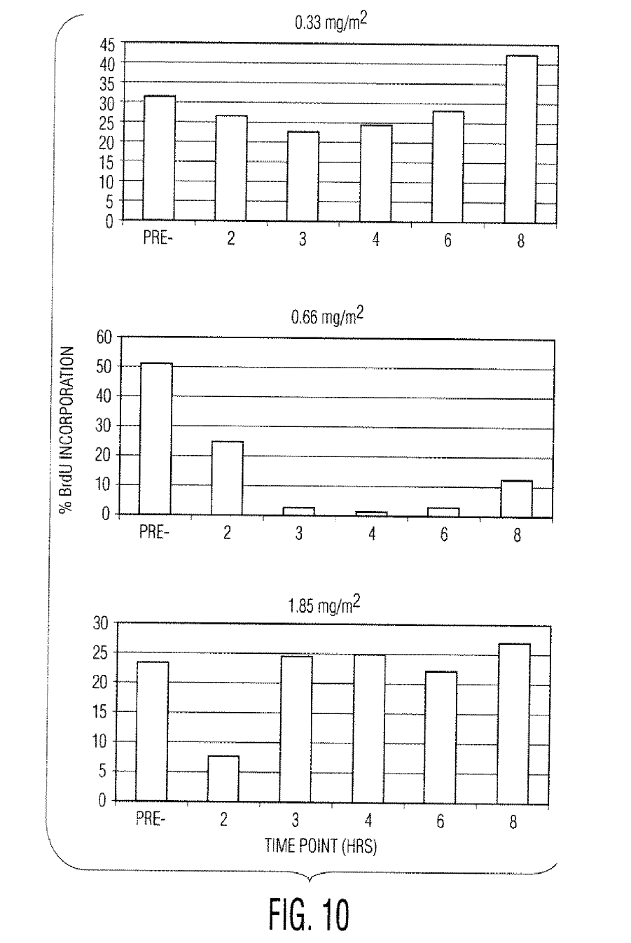

With reference to FIG. 8, which shows mean AUC data for a patient dosed at 1.85 mg/m² on days 1 and 15 of a 28 day dosing schedule, the AUC data for each dose in a cycle are similar. The AUC ratio, expressed as AUC-day 15/AUC-day 1 was 1.09. Plasma concentrations at the end of each infusion on days 1, 8, and 15 of cycle 1, and day 1 of cycle 2 were also found to be similar, indicating consistent exposure for the dosing regime. This indicates also that drug accumulation is not evident using this regime.

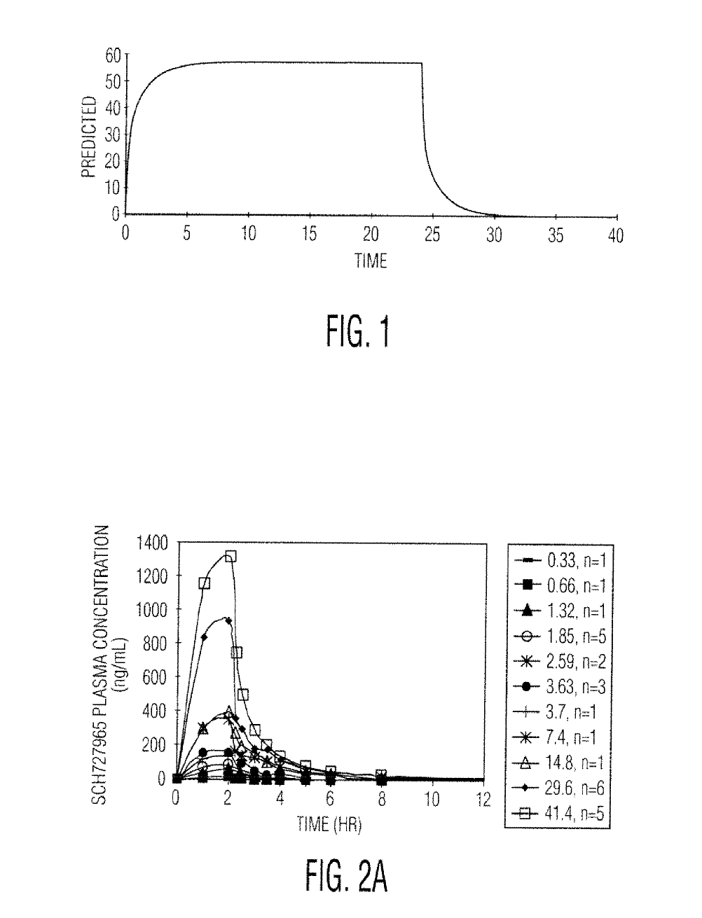

With reference to FIG. 1, which presents an estimate of a concentration/time profile for a 24 hour infusion of a 14.8 mg/m² dose of the compound of Formula II, it is predicted that by extending infusion times to up to 24 hours, a steady state plasma concentration can be established within 2 hours of commencing the infusion and extended over the duration of the infusion. Accordingly, by changing IV infusion time, the PK profile of the compound of Formula II in the present formulation can be altered. In this case maintaining a therapeutically effective plasma level for an extended period of time.

PK parameters observed in these studies are summarized in Table V, Table VI, and Table VII. Specifically, the data shown graphically in FIGS. 2A and 2B correspond to Table V and the data shown graphically in FIG. 2C correspond to Tables VI and VII. These data indicated that PK exhibited moderate inter-subject variability (Cₘₐₓ and AUC) and relatively low to moderate intra-subject variability (approximately 30%) at dosages of from about 0.33 mg/m² to about 58 mg/m². Observed t_{1/2} was between about 1.4 hours and about 3.3 hours.

Table VIII is a cumulative representation of the data in Tables V, VI and VII with an expansion in patient numbers at certain doses as indicated in Table VIII.

TABLE V

| Dosage | t_{1/2} | Cₘₐₓ | AUC | CL |
|---|---|---|---|---|
| (mg/M²) | (hours) | (ng/mL) | (ng hr/mL) | (L/(hr m²) |
| 0.33 | 2.01 | 11.1 | 25.2 | 13.1 |
| 0.66 | 1.48 | 18.6 | 32.1 | 20.6 |
| 1.32 | NA | 63.8 | NA | NA |
| 1.85 | 2.29 (45) | 95.7 (26) | 201.2 (38) | 10.2 (36) |
| 2.59 | 3.0 (1) | 109 (11) | 213 (35) | 12.9 (35) |
| 3.63 | 2.56 (38) | 163 (63) | 423 (91) | 14.7 (91) |
| 3.7 | 2.75 | 136 | 336 | 11.0 |
| 7.4 | 2.86 | 353 | 972 | 7.62 |
| 14.8 | 2.78 | 396 | 1053 | 14.1 |
| 29.6 | 2.98 (11) | 890 (44) | 2434 (45) | 14.0 (40) |
| 41.4 | 2.80 (29) | 1343 (48) | 3041 (51) | 28.3 (133) |

TABLE VI

| | | Cmax | | Tmax | AUC(tf) | | tf | |
|---|---|---|---|---|---|---|---|---|
| Dose | | (ng/mL) | | (hr) | (hr*ng/mL) | | (hr) | |
| (mg/m²) | N | Mean | CV % | Median | Mean | CV % | Median | Range |
| 1.85 | 1 | 62.5 | NA | 1 | 134 | NA | 8 | 8-8 |
| 3.7 | 1 | 136 | NA | 2 | 336 | NA | 24 | 24-24 |
| 7.4 | 1 | 353 | NA | 2 | 970 | NA | 24 | 24-24 |
| 14.8 | 1 | 396 | NA | 2 | 1050 | NA | 24 | 24-24 |
| 29.6 | 6 | 890 | 44 | 1 | 2040 | 57 | 24 | 2.25-24 |
| 41.4 | 6 | 1300 | 45 | 2 | 2870 | 50 | 24 | 6-24 |
| 50 | 10 | 1820 | 49 | 1.5 | 4050 | 63 | 24 | 8-24 |
| 58 | 3 | 1460 | 20 | 1 | 3290 | 36 | 24 | 24-24 |

NA: not applicable

TABLE VII

| | | AUC(I) | | t½ | | CL | |
|---|---|---|---|---|---|---|---|
| Dose | | (hr*ng/mL) | | (hr) | | (L/hr/m²) | |
| (mg/m²) | N | Mean | CV % | Mean | CV % | Mean | CV % |
| 1.85 | 1 | 136 | NA | 1.42 | NA | 13.6 | NA |
| 3.7 | 1 | 336 | NA | 2.75 | NA | 11.0 | NA |
| 7.4 | 1 | 972 | NA | 2.86 | NA | 7.62 | NA |
| 14.8 | 1 | 1050 | NA | 2.78 | NA | 14.1 | NA |
| 29.6 | 5^{a} | 2430 | 45 | 2.98 | 11 | 14.0 | 40 |
| 41.4 | 6 | 2890 | 50 | 2.84 | 26 | 26.8 | 126 |
| 50 | 10 | 4090 | 64 | 2.90 | 29 | 16.3 | 50 |
| 58 | 3 | 3300 | 37 | 3.15 | 15 | 19.7 | 44 |

NA: not applicable;
^{a}n = 4 for t/½ as value not determinable for one patient

TABLE VIII
Mean (CV %) Preliminary Pharmacokinetic Parameters on Day 1 Following
a 2-hour Intravenous Infusion Based on Cumalative Data

| | | Cmax | | Tmax | AUC(I) | | t½ | | CL | |
|---|---|---|---|---|---|---|---|---|---|---|
| Dose | | (ng/mL) | | (hr) | (hr*ng/mL) | | (hr) | | (L/hr/m²) | |
| (mg/m²) | N | Mean | CV % | Median | Mean | CV % | Mean | CV % | Mean | CV % |
| 0.33 | 1 | 11.1 | NA | 1 | 25.2 | NA | 2.01 | NA | 13.1 | NA |
| 0.66 | 1 | 18.6 | NA | 2 | 32.1 | NA | 1.48 | NA | 20.6 | NA |
| 1.32 | 1 | 63.8 | NA | 2 | NA | NA | NA | NA | NA | NA |
| 1.85 | 5^{a} | 91.0 | 26 | 2 | 201 | 38 | 2.29 | 45 | 10.2 | 36 |
| 2.59 | 3^{b} | 124 | 22 | 1 | 213 | NA | 3.02 | NA | 12.9 | NA |
| 3.63 | 7^{c,d} | 195 | 43 | 1 | 446 | 52 | 2.63 | 20 | 11.3 | 69 |
| 3.7 | 1 | 136 | NA | 2 | 336 | NA | 2.75 | NA | 11.0 | NA |
| 5.08 | 3 | 173 | 39 | 2 | 327 | 21 | 3.19 | 7 | 15.9 | 19 |
| 7.11 | 1 | 347 | NA | 1 | 588 | NA | 3.16 | NA | 12.1 | NA |
| 7.4 | 1 | 353 | NA | 2 | 972 | NA | 2.86 | NA | 7.62 | NA |
| 14.8 | 1 | 396 | NA | 2 | 1050 | NA | 2.78 | NA | 14.1 | NA |
| 29.6 | 6^{e} | 890 | 44 | 1 | 2430 | 45 | 2.98 | 11 | 14.0 | 40 |
| 41.4 | 6 | 1300 | 45 | 2 | 2890 | 50 | 2.84 | 26 | 26.8 | 126 |
| 50 | 10 | 1820 | 49 | 1.5 | 4090 | 64 | 2.90 | 29 | 16.3 | 50 |
| 58 | 3 | 1460 | 20 | 1 | 3300 | 37 | 3.15 | 15 | 19.7 | 44 |

Abbreviations: AUC(I) = area under the plasma concentration-time curve from time zero to infinity; CL = clearance; Cmax = maximum observed plasma concentration; CV % = coefficient of variation, expressed in percent; t½ = terminal phase half life; Tmax = time to maximum observed plasma concentration; NA = not applicable
^{a}N = 4: AUC(I), t½ and CL; t½ were not determinable for a patient
^{b}N = 2: AUC(I), t½ and CL; t½ were not determinable for a patient
^{c}N = 6: AUC(I), t½ and CL; t½ were not determinable for a patient
^{d}Data from a patient was not included in descriptive statistics, patient received a <1 hr infusion and <3.63 mg/m² dose
^{e}N = 5: AUC(I), t½ and CL; t½ were not determinable for a patient

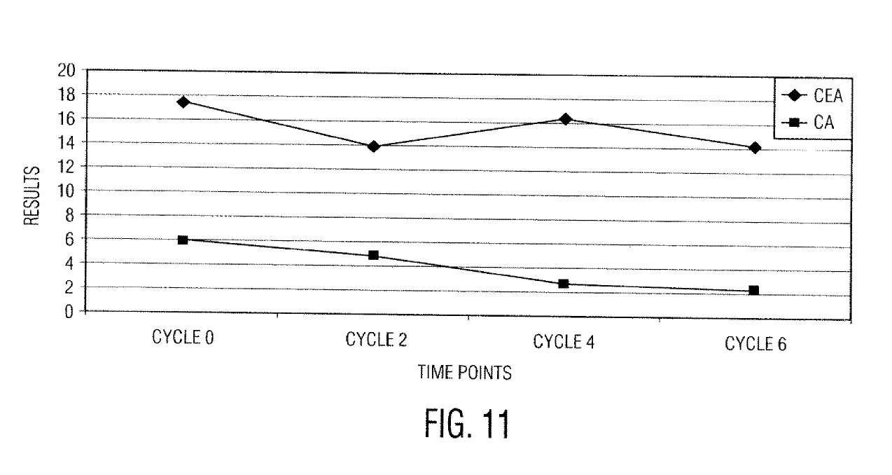

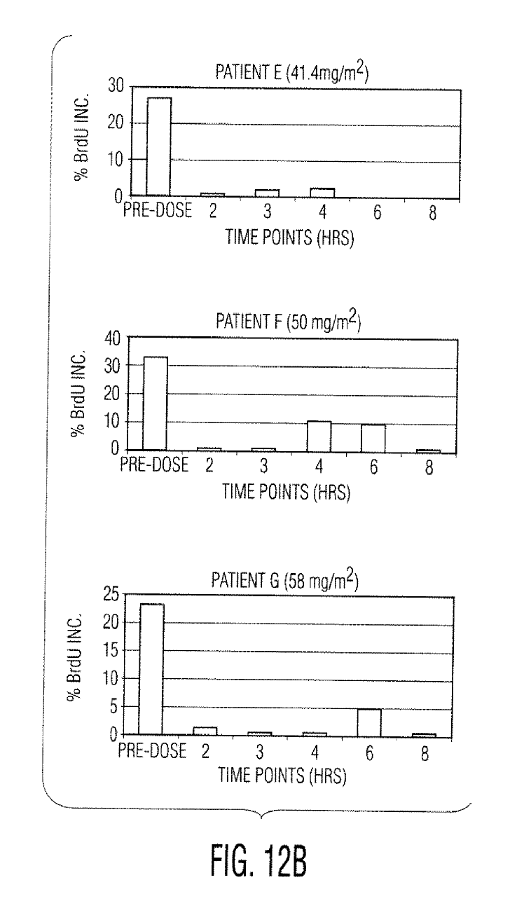

Blood samples were collected at 2, 3, 4, 6, and 8 hours post initiation of infusion from patients infused (2 hrs) with dosage levels of the compound of Formula II in amounts equal to 0.33 mg/m²; 0.66 mg/m²; and 1.85 mg/m². The samples were collected in tubes containing heparin and shipped to a central laboratory for evaluation. Whole blood from each sample was diluted 1:5 in complete RPMI media and 200 microliter aliquots were added to wells of a 96-well plate. Samples from each time point were either stimulated with PHA (0.01 mg/ml final concentration), or not stimulated. The sample well plates were incubated for 48 hours at 37° C. under 5% CO₂ and BrdU (10 micromolar) was added to each well. The plates were incubated for an additional 24 hours. Samples were stained with FITC conjugated anti-CD45 antibody. RBC's were lysed and the remaining cells were fixed, permeabilized and treated with DNase. The samples were then incubated with an anti-BrdU antibody. Washed cells were re-suspended and the CD45 and BrdU-positive and −negative populations were detected by FACS. FIGS. 10, 12A, and 12B show the percent of BrdU incorporation for samples taken for various doses.

With reference to FIG. 10, no inhibition of BrdU incorporation is observed at a dose of 0.33 mg/m², but at a dose of 0.66 mg/m², inhibition of BrdU incorporation is observed up to eight hours post infusion. Inhibition of BrdU incorporation is observed at two hours post-infusion at a dose of 1.85 mg/m². These data indicate that the compound of Formula II interacts with cellular mechanistic pathways in a manner likely to disrupt tumor cells.

With reference to FIG. 12A, mild inhibition of BrdU incorporation is observed at two hours post-infusion at a dose of 1.85 mg/m². In contrast, as shown in FIGS. 12A and 12B, inhibition of BrdU incorporation is observed up to 3 hours post infusion at a dose of 3.7 mg/m² and 7.4 mg/m² and up to eight hours post infusion at a dose of 29.6 mg/m², 41.4 mg/m², 50 mg/m², and 58 mg/m². The effect of the compound of Formula II on inhibition of BrdU incorporation observed at a dose of 1.85 mg/m² (see FIG. 12A) compared to that seen in FIG. 10 can be attributed to Inter-subject variability.

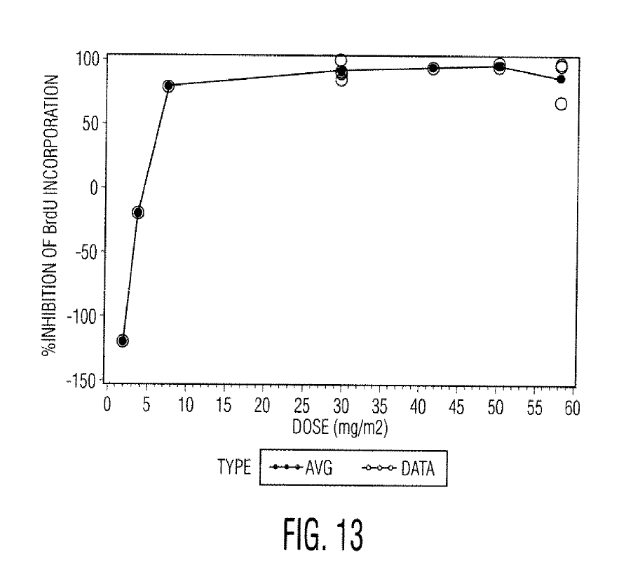

With reference to FIG. 13 and Table IXI, percentage inhibition of BrdU incorporation at 8 hours post-infusion generally increased over the range of 1.85 to 58 mg/m². In fact, the percentage inhibition of BrdU incorporation already rose to greater than 75% at a dose of 7.4 mg/m². These data reflect that inhibition of lymphocyte proliferation as measured by BrdU incorporation correlates with the dosage level of the compound of Formula II to which the blood sample was exposed.

TABLE IX

| | | | | % inhibition of |
|---|---|---|---|---|
| | | | % BrdU | BrdU |
| | | % BrdU | incorporated at | incorporation at |
| | Dose | incorporated | 8 hrs post- | 8 hrs post- |
| Patient | (mg/M2) | Pre-Dose | infusion | infusion |
| A | 1.85 | 28 | 65 | −133 |
| B | 3.7 | 10 | 13 | −25 |
| C | 7.4 | 51 | 11 | 79 |
| D | 29.6 | 29 | 3 | 92 |
| E | 41.4 | 27 | 2* | 97* |
| F | 50 | 33 | 1 | 98 |
| G | 58 | 23 | 0.7 | 98 |
| H | 41.4 | 41 | 45 | −10 |

*Values at 4 hr post-infusion

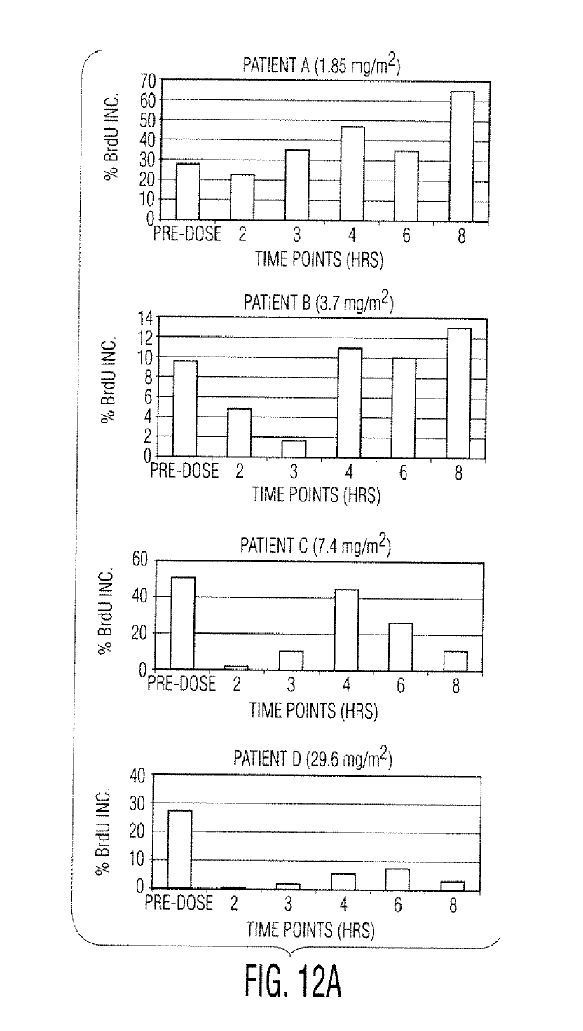

Patients having a diagnosis of non small cell lung cancer (NSCLC), colorectal cancer, prostate cancer, ovarian cancer, breast cancer, melanoma, vulvar cancer, pseudomyxoma peritonei, peritoneal cancer, cholangiocarcinoma, pancreatic cancer, soft tissue sarcoma, gastric cancer, gastrointestinal stromal tumor (GIST), esophageal cancer, adenoidcystic carcinoma, neuroendocrine tumor or hepatocellular carcinoma have been administered various dosages of the compound of Formula II by 2 hour IV infusion on a 28-day dosing cycle. Each 28-day dosing cycle comprising once weekly administration for three weeks with a week off. No complete or partial responses have been observed by RECIST criteria in the population (see, e.g., Therasse et al., J Natl Cancer Inst, 92(3):205-216 (2000)). Nonetheless, one patient diagnosed with NSCLC has stable disease after 6 cycles of 28 day treatment at 0.66 mg/m² and one patient diagnosed with pseudomyxoma peritonei has falling tumor markers, as shown in FIG. 11, after 6 cycles of 28 day treatment at 2.59 mg/m², and continued on treatment for a total of 9 cycles For clarity, the tumor marker indicated as “CA” is CA125. Additionally, one patient with prostate cancer received 4 cycles of 28-day dosing regime who was dosed at 3.63 mg/m². One patient with melanoma was treated at 3.63 mg/m2 for 9 cycles until disease progression. One patient with soft tissue sarcoma receiving 6 cycles at 7.11 mg/m2 continues to have stable disease as does one patient with GIST receiving 5 cycles at 7.11 mg/m2. One patient with esophageal cancer receiving 5 cycles at 7.11 mg/m2 has stable disease. One patient with adenoidcystic carcinoma received 5 cycles at 7.11 mg/m2 until disease progression.

Likewise, patients having a diagnosis of a carcinoid, thymic cancer, leiomysarcoma, NSCLC, colorectal cancer, squamous cell carcinoma of the head and neck (HNSCC), adenocarcinoma of unknown primary, melanoma, ovarian cancer, non-Hodgkin's lymphoma, neuroendocrine tumor, testicular cancer, breast cancer, prostate cancer, esophageal cancer, pancreatic cancer, thyroid cancer, liposarcoma, cholangiocarcinoma, pseudomyxoma peritonei, chordoma, or sarcomatoid carcinoma were administered various dosages of the compound of Formula II by 2 hour IV infusion on a 21-day dosing cycle. Patients having a diagnosis of liposarcoma, colorectal cancer, neuroendocrine tumor, NSCLC, and esophageal cancer were administered various dosages of the compound of Formula II by 8 hour IV infusion on a 21-day dosing cycle. Each 21-day dosing cycle comprising administration of the dose on Day 1 of a 21-day period Although no complete or partial responses were observed by RECIST criteria in the population, stable disease was achieved in some of the aforementioned tumor types after 4 or more 21-day dosing cycles. Specifically, two carcinoid patients achieved a stable disease state for 4 cycles at a dose of 29.6 mg/m² and 41.4 mg/m². Similarly, a thymic cancer patient achieved a stable disease state for 4 cycles at a dose of 50 mg/m². A patient with anal cancer achieved stable disease for 6 cycles at a dose of 50 mg/m2. Additionally, a leiomysarcoma patient achieved a stable disease state for 8 cycles at a dose of 41.4 mg/m². A NSCLC patient achieved a stable disease state for 12 cycles at a dose of 50 mg/m². A chordoma patient achieved stable disease for 8 cycles at a dose of 58 mg/m2 and a prostate cancer patient achieved stable disease for 7 cycles at a dose of 50 mg/m2.

The above description, of the invention is intended to be illustrative and not limiting. Various changes or modifications in the embodiments described herein may occur to those skilled in the art. These changes can be made without departing from the scope or spirit of the invention

TABLE X
4-week schedule

| Tumor | Dose mg/m² | Cycle no. | Status |
|---|---|---|---|
| NSCLC | 0.66 | 6 | off study with stable disease |
| pseudomyxoma | 2.59 | 9 | off study with stable disease |
| prostate | 3.63 | 4 | disease progression |
| melanoma | 3.63 | 9 | disease progression |
| soft tissue | 7.11 | 6 | on treatment |
| sarcoma | | | |
| GIST | 7.11 | 5 | on treatment |
| esophageal ca | 7.11 | 5 | on treatment |
| adenoidcystic | 7.11 | 5 | disease progression |

TABLE XI
21 day schedule

| Tumor | Dose mg/m² | Cycle no. | Status |
|---|---|---|---|
| carcinoid | 29.6 | 4 | disease progression |
| carcinoid | 41.4 | 4 | disease progression |
| leiomyosarcoma | 41.4 | 8 | disease progression |
| thymic | 50 | 4 | disease progression |
| NSCLC | 50 | 12 | on treatment |
| chordoma | 58 | 8 | on treatment |
| anal cancer | 50 | 6 | disease progression |
| prostate | 50 | 7 | on treatment |

The above description of the invention is intended to be illustrative and not limiting. Various changes or modifications in the embodiments described herein may occur to those skilled in the art. These changes can be made without departing from the scope or spirit of the invention

1. An IV-infusible formulation comprising the compound of Formula II in a buffered solution having a pH of from about pH 3.4 to about pH 5, the formulation providing at least one PK behavior and/or AUC in accordance with that shown in either FIG. 2A, 2B, or 2C when infused into a human in at least one of the dosage levels indicated.

2. The formulation of claim 1 which provides the AUC/dose curves illustrated in FIG. 3 when infused in a two hour infusion in an amount providing at least one dosage level shown.

3. The formulation of claim 1 wherein the buffered solution is an aqueous buffered solution comprising an acid and its conjugate sodium salt wherein the acid is citric acid or lactic acid.

4. The formulation of claim 1 wherein the buffered solution comprises citric acid and sodium citrate.

5. The formulation of claim 4 wherein the ratio of citric acid:sodium citrate in the buffered solution is about 1 mole of sodium citrate to about 2.4 moles of citric acid.

6. The formulation of claim 5 wherein the solution additionally comprises a sodium chloride solution diluent.

7. A method of treating a tumor condition treatable by the administration of the compound of Formula II comprising infusing a solution comprising the compound of Formula II into a patient in need of treatment over a period of from 1 hour to about 24 hours on each of days 1, 8, and 15 of a 28 day cycle, in an amount effective to treat the tumor condition.

8. The method of claim 7 wherein the cycle is repeated until tumor inhibition or complete remission is observed.

9. The method of claim 7 wherein the amount of the solution infused provides a plasma level of at least about 10 ng/mL.

10. The method of claim 7 wherein the solution administered comprises a buffered solution comprising citric acid and sodium citrate, wherein the ratio of citric acid:sodium citrate in the buffered solution is about 1 mole of sodium citrate to about 2.4 moles of citric acid.

11. A process for making a concentrate comprising preparing a buffered solution of an organic acid and its conjugate salt, dissolving the compound of Formula II in the buffered solution and adjusting the pH of the resulting solution to a pH of from about pH 3 to about pH 5.1.

12. The process of claim 11 which further includes the step of admixing the concentrate with the contents of a standard saline IV bag, thereby providing an infusible formulation.

13. The process of claim 11 wherein the organic acid is citric acid and the conjugate salt is sodium citrate.

14. The process of claim 11 wherein the ratio of organic acid, conjugate salt and the compound of Formula II is 1 mole of the compound of Formula II: 3.66 moles conjugate salt:8.8 moles organic acid.

15. The process of claim 11 wherein the compound of Formula II is present in the concentrate at the level of 5 mg/mL.

16. A formulation comprising an aqueous organic acid, its conjugate salt, and the compound of Formula II which provides the PK profile shown in FIG. 2 A upon IV infusion into a human.

17. A formulation comprising an aqueous organic acid, its conjugate salt, and the compound of Formula II which provides at least one of the PK profiles shown in Table V upon IV infusion into a human in an amount providing at least one of the indicated dosages.

18. The formulation of claim 17 comprising a mole ratio of the compound of Formula II: organic acid:conjugate salt of 1 mole:8.8 moles:3.7 moles.

19. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 1.85 mg/M² having a t_{1/2} of about 2.15 hours, a Cₘₐₓ of about 77 ng/ml, and/or an AUC of about 181 ng hr/mL.

20. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 33 mg/M² having a t_{1/2} of about 2.75 hours, a Cₘₐₓ of about 136 ng/ml, and/or an AUC of about 336 ng hr/mL.

21. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 7.4 mg/M² having a t_{1/2} of about 2.86 hours, a Cₘₐₓ of about 353 ng/ml, and/or an AUC of about 994 ng hr/mL.

22. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 14.8 mg/M² having a t_{1/2} of about 2.78 hours, a Cₘₐₓ, of about 396 ng/ml, and/or an AUC of about 1053 ng hr/mL.

23. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 29.6 mg/M² having a t_{1/2} of about 2.79 hours, a Cₘₐₓ of about 1020 ng/ml, and/or an AUC of about 2531 ng hr/mL.

24. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 41.4 mg/M² having a t_{1/2} in the range of about 2 to about 2.8 hours, a Cₘₐₓ in the range of about 1020 ng/ml to 1343 ng/ml, and/or an AUC in the range of about 2531 ng hr/mL to about 3041 ng hr/mL.

25. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 50 mg/M² having a t_{1/2} of about 1.5 hours, a Cₘₐₓ of about 1820 ng/ml, and/or an AUC of about 3290 ng hr/mL.

26. A formulation comprising a mole ratio of the compound of Formula II: organic acid:conjugate salt of about 1 mole of the compound of Formula II: about 8.8 moles of organic acid:about 3.7 moles of conjugate salt, wherein, the formulation provides a PK for a two hour infusion of a dose comprising 58 mg/M² having a t_{1/2} of about 1 hours, a Cₘₐₓ of about 1460 ng/ml, and/or an AUG of about 3290 ng hr/mL.

27. A formulation comprising the compound of Formula II exhibiting the PK values and Cₘₐₓ value shown in Table V when administered by an IV infusion in an amount providing the corresponding dose indicated in Table V.

28. A formulation comprising the compound of Formula II exhibiting the PK values and Cₘₐₓ value shown in Table VI when administered by an IV infusion in an amount providing the corresponding dose indicated in Table VI.

29. A method of treating a tumor condition treatable by the administration of the compound of Formula II comprising infusing a solution comprising the compound of Formula II into a patient in need of treatment over a period of from 1 hour to about 24 hours wherein the compound is administered on Day 1 followed by 20 days off (21 day cycle), in an amount effective to treat the tumor condition.

30. The method of claim 29 wherein the cycle is repeated until tumor inhibition or complete remission is observed.

31. The method of claim 29 wherein the amount of the solution infused provides a plasma level of at least about 10 ng/mL.

32. The method of claim 29 wherein the solution administered comprises a buffered solution comprising citric acid and sodium citrate, wherein the ratio of citric acid:sodium citrate in the buffered solution is about 1 mole of sodium citrate to about 2.4 moles of citric acid.

33. The method of claim 7 wherein the amount of solution infused provides up to about 58 mg/m² of the compound of Formula II.

34. The method of claim 7 wherein the amount of solution infused provides about 50 mg/m² of the compound of Formula II.

35. The method of claim 29 wherein the tumor is selected from the group consisting of non-small cell lung cancer (NSCLC), breast cancer, ovarian cancer, acute leukemias, chronic leukemias (e.g., CLL), non-Hodgkin's lymphoma (e.g., MCL), melanoma, and multiple myeloma.

36. The method of claim 29 wherein the time period for the infusion is selected from the group consisting 2 hours, 8 hours and 24 hours.

37. The method of claim 29, wherein the amount of compound of Formula H administered is from about 0.33 mg/m² to about 20 mg/m², from about 0.33 mg/m² to about 14 mg/m² or from about 7 mg/m² to about 12 mg/m².

38. A method of treating a tumor condition treatable by the administration of the compound of Formula II comprising infusing a solution comprising the compound of Formula II into a patient in need of treatment over a period of from 1 hour to about 24 hours wherein the compound is administered on each of days 1, 8, and 15 of a 28 day cycle, in an amount effective to treat the tumor condition.

39. The method of claim 29 wherein the tumor is selected from the group consisting of non-small cell lung cancer (NSCLC), breast cancer, ovarian cancer, acute leukemias, chronic leukemias (e.g., CLL), non-Hodgkin's lymphoma (e.g., MCL), melanoma, and multiple myeloma.

40. The method of claim 38 wherein the time period for the infusion is selected from the group consisting 2 hours, 8 hours and 24 hours.

41. The method of claim 40 wherein the time period for the infusion is 8 hours.

42. The method of claim 41, wherein the amount of compound of Formula II administered is from about 1.85 mg/m² to about 70 mg/m² or from about 15 mg/m² to about 30 mg/m².

43. The method of claim 40 wherein the time period for the infusion is 24 hours.

44. The method of claim 43, wherein the amount of compound of Formula II administered is from about 1.85 mg/m² to about 100 mg/m², from about 50 mg/m² to about 80 mg/m² or from about 30 mg/m² to about 70.0 mg/m².

45. The method of claim 38, wherein solid tumors are treated.

46. The method of claim 45, wherein the solid tumors are selected from the group consisting of breast, melanoma and ovarian cancer.

47. The method of claim 45, wherein the amount of solution infused provides from about 1.8 mg/m² to about 58 mg/m².

48. The method of claim 45 further comprising the administration of growth factors.

49. The method of claim 48 wherein the growth factors are selected from the group consisting of GCSF and GMCSF.

50. The method of claim 48 wherein the amount of solution infused provides between about 0.33 mg/m² and from about 100 mg/m² in a 21 day schedule.

51. A formulation comprising an aqueous organic acid, its conjugate salt, and the compound of Formula II which provides the PK profile shown in FIG. 2B upon IV infusion into a human.

52. A formulation comprising an aqueous organic acid, its conjugate salt, and the compound of Formula II which provides the PK profile shown in FIG. 2C upon IV infusion into a human.

53. The method of claim 29 wherein the amount of solution infused provides up to about 58 mg/m² of the compound of Formula II.

54. The method of claim 29 wherein the amount of solution infused provides about 50 mg/m² of the compound of Formula II.

55. The method of claim 46 further comprising the administration of growth factors.

56. The method of claim 55 wherein the growth factors are selected from the group consisting of GCSF and GMCSF.

57. The method of claim 49 wherein the amount of solution infused provides between about 0.33 mg/m² and from about 100 mg/m² in a 21 day schedule.
